(19) United States
(12) Patent Application Publication
(10) Pub. No.: US 2023/0419371 A1
(43) Pub. Date: Dec. 28, 2023

(54) SYSTEM AND METHOD FOR CONNECTING PEOPLE WITH SERVICE PROVIDERS IN REAL-TIME

(71) Applicant: QDoc Inc., Winnipeg (CA)

(72) Inventors: Norman Silver, Winnipeg (CA); David Berkowits, Winnipeg (CA)

(21) Appl. No.: 18/179,468

(22) Filed: Mar. 7, 2023

Related U.S. Application Data
(60) Provisional application No. 63/370,955, filed on Aug. 10, 2022.
(60) Provisional application No. 63/366,828, filed on Jun. 22, 2022.

(51) Int. Cl. G06Q 30/02; G16H 80/00; G06Q 50/20

(52) U.S. Cl. CPC G06Q 30/0281; G06Q 50/205; G16H 80/00

(57) ABSTRACT

Disclosed is a method for execution by a server for connecting people with service providers in real-time. The server maintains a set of rules for connecting people with service providers. The server receives, from a first client computing device over a network, a request message having personal information identifying at least a personal issue. The server assesses the personal information against the set of rules to identify service providers that can address the personal issue. Next, for a first subset of service providers, the server sends offer messages and waits for an acceptance message. However, if no acceptance message is received within a timeout period, for a subsequent subset of service providers, the server sends offer messages and waits for an acceptance message. Advantageously, if no match can be established with the first subset, it may be possible to establish a match with the subsequent subset.

### CROSS-REFERENCE TO RELATED APPLICATIONS

This application claims the benefit of U.S. provisional patent application Ser. No. 63/370,955, filed Aug. 10, 2022, U.S. provisional patent application Ser. No. 63/366,828, filed Jun. 22, 2022, and U.S. provisional patent application Ser. No. 63/362,091, filed Mar. 29, 2022, each of which are incorporated herein by reference.

### FIELD OF THE DISCLOSURE

This disclosure relates to communication systems, and more particularly to communication systems for connecting people to service providers.

### BACKGROUND

In general, connecting people to service providers in a timely manner can be challenging and inefficient. There are several issues at play such as suitability, availability, geographic location, and timing. Issues may be present with both the people wanting service and the service providers too.

As a first example, connecting patients to physicians or other health care professionals in an on demand environment presents many challenges. A patient having a particular ailment such as a rash for example might first see a GP (General Practitioner) who might then refer the patient to see a Dermatologist who specializes in rashes and other skin issues. However, having an initial meeting with the GP, and then having a subsequent meeting with the Dermatologist can take time. For example, it is possible that months can pass before the patient sees the Dermatologist, because the Dermatologist could be busy with a full patient load. Meanwhile, it may be possible that another Dermatologist who is nearby has availability to see the patient much earlier, but often such opportunities are not known and thus not exploited. As a result, the patient may end up waiting a long time to see the Dermatologist whilst suffering with the rash.

As a second example, connecting students to tutors or other teaching professionals presents many challenges. A student having a particular academic issue, such as difficulty with grade 12 chemistry for example, might first look for a tutor who can teach chemistry. However, finding a suitable tutor with availability in vicinity of the student can be difficult. The student may end up having to travel long distances to find a suitable tutor or wait a long time to meet with a suitable tutor in closer vicinity.

The conventional approaches of connecting people to service providers in a timely manner leave much to be desired. It is desirable to improve upon the conventional approaches by employing technology to eliminate or mitigate some or all of the aforementioned shortcomings.

### SUMMARY OF THE DISCLOSURE

Disclosed is a method for execution by a server for connecting people with service providers in real-time. The server maintains a set of rules for connecting people with service providers. Note that these rules are application-specific. The server receives, from a first client computing device over a network, a request message having personal information identifying at least a personal issue. The server then assesses the personal information against the set of rules to identify a first subset of service providers that can address the personal issue. For each service provider of the first subset, the server sends an offer message over the network to a client computing device of the service provider.

In accordance with an embodiment of the disclosure, if an acceptance message is not received within a first timeout period, the server identifies, based on the set of rules, a subsequent subset of service providers that can address the personal issue, such that the subsequent subset includes at least one additional service provider not included in any preceding subset identified for the request message. Then, for each additional service provider of the subsequent subset, the server sends a subsequent offer message over the network to a client computing device of the additional service provider. If an acceptance message is received within a subsequent timeout period, then the server sends a response message to the first client computing device over the network, such that the response message indicates acceptance.

The first subset of service providers may include favoured service providers according to some criteria, while the subsequent subset of service providers may include other service providers that are less favoured but will nonetheless suffice. In this way, if no match can be established with a favoured service provider, it may still be possible to establish a match with another service provider that will suffice, thereby connecting a person with a suitable service provider. Such connection occurs in real-time with practical application of network technology and hence would not be possible without the network technology as described herein. Such technology places the service providers in competition with one another to accept offer, thereby mitigating wait time for the person. To the extent that subsequent interaction is desirable, such as interaction for arranging a specific time slot for meeting, the person can make such arrangement directly with the service provider. Alternatively, some of these arrangements could be addressed automatically according to some implementations disclosed herein.

In some implementations, the personal information is patient information with the personal issue being a medical issue, and each service provider is a physician or other medical professional. In such case, the personal information may include a patient location, and the service providers identified for the first subset may include only service providers in vicinity of the patient location. In other implementations, the personal information is student information with the personal issue being an academic issue, and each service provider is a tutor or other teaching professional. In such case, the service providers identified for the first subset may include only service providers that are experts with the academic issue. Other applications for connecting people with service providers in real-time are possible and are within the scope of the disclosure.

Also disclosed is a method for execution by a server for connecting people with service providers in real-time. The method involves receiving, from a first client computing device over a network, a request message having a code, and identifying a service provider associated with the code. The method also involves sending an offer message over the network to a client computing device of the service provider, and if an acceptance message is received over the network within a timeout period, sending a response message to the first client computing device over the network, such that the response message indicates acceptance.

Also disclosed is a non-transitory computer readable medium having recorded thereon statements and instructions that, when executed by a processor of a server, configure the server to implement the method summarized above.

Also disclosed is a server configured to connect people with service providers in real-time. The server has a network adapter, and rules engine circuitry coupled to the network adapter and configured to implement the method summarized above.

Other aspects and features of the present disclosure will become apparent, to those ordinarily skilled in the art, upon review of the following description of the various embodiments of the disclosure.

### BRIEF DESCRIPTION OF THE DRAWINGS

Embodiments will now be described with reference to the attached drawings in which:

FIG. 1 is a block diagram of a communication system having a server coupled to a plurality of client computing devices via a network;

FIG. 2 is a flowchart of a method of connecting people to service providers in real-time;

FIG. 3 is a flowchart of a method of connecting a person to a service provider using a code;

FIG. 4 is a schematic of a rules based engine for connecting patients to physicians in real-time; and

FIG. 5 is a schematic of example scenarios of connecting people to service providers using codes.

### DETAILED DESCRIPTION OF EMBODIMENTS

It should be understood at the outset that although illustrative implementations of one or more embodiments of the present disclosure are provided below, the disclosed systems and/or methods may be implemented using any number of techniques. The disclosure should in no way be limited to the illustrative implementations, drawings, and techniques illustrated below, including the exemplary designs and implementations illustrated and described herein, but may be modified within the scope of the appended claims along with their full scope of equivalents.

### Introduction

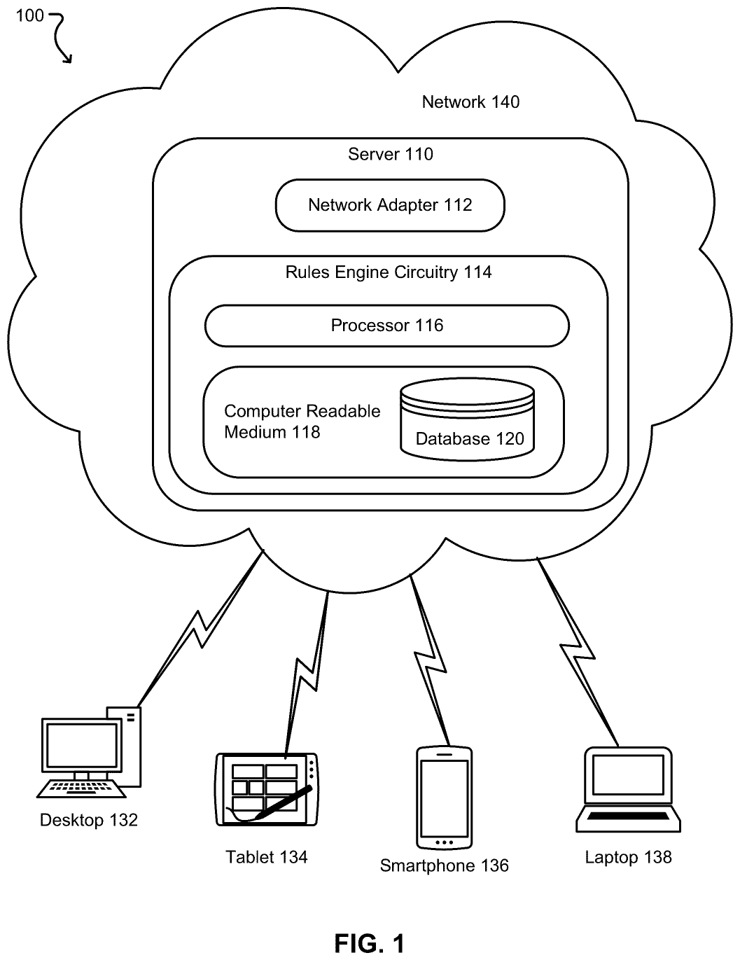

Referring now to FIG. 1, shown is a block diagram of a communication system 100 having a server 110 coupled to a plurality of client computing devices 132,134,136,138 via a network 140. The communication system 100 can have other components as well, but these are not shown for simplicity. The server 110 has a network adapter 112 for communicating with the client computing devices 132,134,136,138 over the network 140. The server 110 also has rules based engine circuitry 114. The server 110 can have additional components, but these are not shown for simplicity.

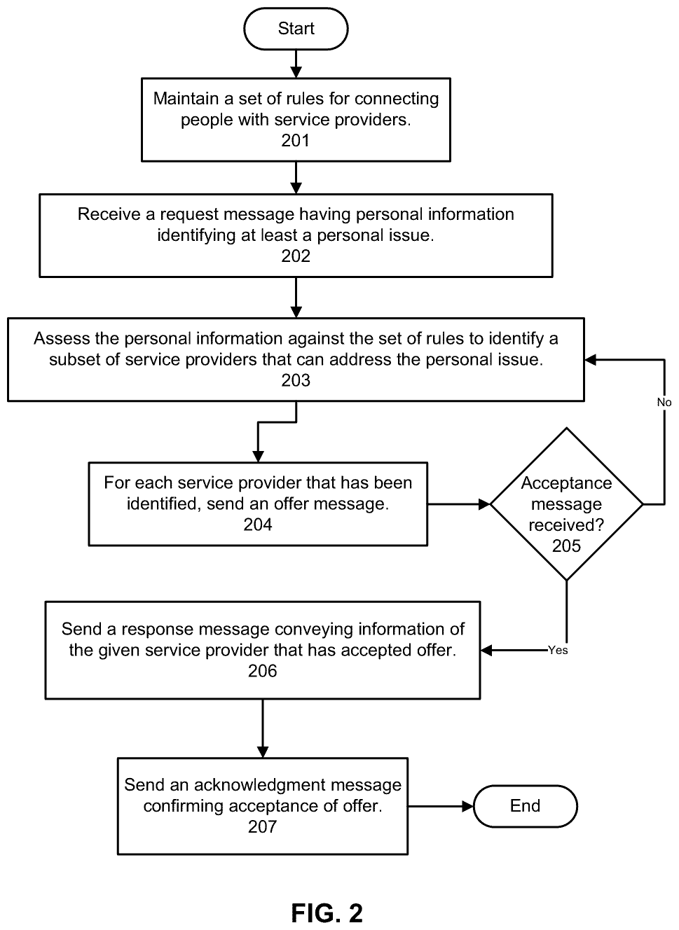

The rules based engine circuitry 114 of the server 110 operates to connect people to service providers in real-time, in accordance with a defined set of rules. Such operation will be described below with reference to FIG. 2, which is a flowchart of a method of connecting people to service providers in real-time. Although the method of FIG. 2 is described below with reference to the server 110 in the communication system 100 shown in FIG. 1, it is to be understood that the method of FIG. 2 is applicable to other systems. In general, the method of FIG. 2 is applicable to the server 110 in any appropriately configured system.

At step 201, the server 110 maintains a set of rules for connecting people with service providers. Note that these rules are application-specific. Example rules will be described later. At step 202, the server 110 receives, from a first client computing device 132 over a network 140, a request message including personal information identifying at least a personal issue. In some implementations, the personal information also identifies additional information such as location, age, sex, prior encounter, spoken language, etc.

At step 203, the server 110 assesses the personal information against the set of rules to identify a first subset of service providers that can address the personal issue. Note that the first subset can include one or more service providers. The first subset of service providers normally include favoured service providers according to some criteria, which could be based on the personal information that has been supplied. For example, if the personal information includes a location, then the first subset of service providers identified at step 203 might include only service providers in vicinity of the location that can address the personal issue. Other possibilities exist depending on what personal information is supplied.

At step 204, for each service provider of the first subset, the server 110 sends an offer message over the network 140 to a client computing device 134 of the service provider. In some implementations, the offer message includes at least some of the personal information, which might help the service providers to decide whether to accept. In other implementations, the offer message does not include the personal information, because the server 110 has already determined that the service providers of the first subset are suitable and so decisions by the service providers could be as simple as whether they have availability. At step 205, the server 110 waits for a first timeout period stemming from the offer message to see if an acceptance message is received from any of the service providers of the first subset.

In accordance with an embodiment of the disclosure, if no acceptance message is received within the first timeout period, then the server 110 repeats steps 203 and 204 until such acceptance message is received within a subsequent timeout period. In doing so, the scope of the service providers identified can be expanded, for example to include additional service providers that are farther away and/or additional service providers that are otherwise less than ideal. In particular, for each iteration, the server 110 will identify a subsequent subset of service providers that can address the personal issue, such that the subsequent subset will include at least one additional service provider not included in any preceding subset.

The first subset of service providers may include favoured service providers according to some criteria, while the subsequent subset of service providers may include other service providers that are less favoured but will nonetheless suffice. In this way, if no match can be established with a favoured service provider (first iteration), it may still be possible to establish a match with another service provider that will suffice (subsequent iteration), thereby connecting a person with a suitable service provider in the end.

For each additional service provider of the subsequent subset, the server 110 sends a subsequent offer message over the network 140 to a client computing device of the additional service provider. In some implementations, the subsequent subset includes at least one repeat service provider included in a preceding subset, and the server 100 sends the subsequent offer message to each service provider of the subsequent subset including each repeat service provider. For such implementations, some service providers may receive multiple offer messages.

If an acceptance message is received from a service provider within the timeout period, then at step 206 the server 110 sends a response message to the first client computing device 132 over the network 140, such that the response message indicates acceptance. In some implementations, the response message conveys information of the service provider that has accepted offer. In other implementations, information of the service provider are provided through other means.

Upon completion of the method, a person has been connected with a suitable service provider. Such connection occurs in real-time with practical application of network technology and hence would not be possible without the network technology as described herein. Such technology places the service providers in competition with one another to accept offer, thereby mitigating wait time for the person. To the extent that subsequent interaction is desirable, such as interaction for arranging a specific time slot for meeting, the person can make such arrangement directly with the service provider. Alternatively, some of these arrangements could be addressed automatically. For example, in some implementations, the personal information further identifies timing for service (e.g. date and/or time), and the offer message identifies the timing for service. This can be useful for arranging an appointment for the service. Tracking of response time and implementation of an escalation workflow are part of this.

It is noted that the server 110 could receive acceptance messages from multiple service providers during the timeout period. For instance, acceptance messages could be received from three client computing devices 134,136 and 138 in connection with three different service providers. In some implementations, in the case of acceptance messages from multiple service providers, the first acceptance message will determine which service provider is selected. For instance, if the acceptance message is received from the client computing device 134 before the acceptance messages from the other client computing devices 136 and 138, then the service provider associated with the client computing device 134 would be selected. By rewarding the service provider who sends the first acceptance message, there us competition among service providers to encourage the service providers to quickly respond, thereby mitigating or eliminating waiting experienced by people looking for a service provider. In other implementations, another selection criteria is used, such as deciding which service provider is best suited based on some criteria. In some implementations, a combination of both timing and other criteria are used.

In some implementations, at step 207 the server 110 sends, over the network 140 to the client computing device 134 of the given service provider, an acknowledgment message confirming their acceptance of offer. This informs the given service provider that they have been selected. In some implementations, rejection messages are sent to other service providers. For instance, rejection messages could be sent to the other client computing devices 136 and 138 if they sent acceptance messages.

In some implementations, the offer message at step 204 and the acceptance message at step 205 are both text messages. However, other messaging technologies are possible such as email for example. In general, any electronic messaging technology can be employed.

In some implementations, the personal information is patient information with the personal issue being a medical issue, and each service provider is a physician or other medical professional. In such case, the personal information may include a patient location, and the service providers identified for the first subset may include only service providers in vicinity of the patient location. In some implementations, the set of rules includes criteria based on location, age, sex, prior encounter, and medical issue. Further example details and case scenario(s) are provided later.

In other implementations, the personal information is student information with the personal issue being an academic issue, and each service provider is a tutor or other teaching professional. In such case, the service providers identified for the first subset may include only service providers that are experts with the academic issue. In some implementations, the set of rules includes criteria based on location, academic level, and academic issue. Further example details and case scenario(s) are provided later.

Note that other applications are possible beyond medical and academic applications. Thus, the personal information identifying at least a personal issue is not limited medical and academic issues, but rather can include other personal issues. Other personal issues can include a desire for a service, such as a haircut or an oil change for example. Case examples demonstrating this point are provided below. Other applications could include legal applications (e.g. family law) or veterinary medicine for example.

In some implementations, the set of rules include settings, which can be configurable for example by a service provider or by another person such as a system administrator. The server 110 can receive input for modifying the settings, and update the settings in accordance with the input. This can enable the service provider or other person to adjust the set of rules as may be appropriate.

In some implementations, a set of rules concerning a service provider can be configured by the service provider. For example, a physician or a group of physicians could manage a set of rules governing what type of patient they will see (e.g. age, sex, underlying issue, etc.). Additionally, or alternatively, the physician or the group of physicians can make changes to the settings to facilitate follow-up appointments for existing patients, with a view to managing ongoing care or for other reasons. For example, a physician who has an initial appointment with a patient may wish to see that patient on an ongoing basis to manage ongoing care for the patient depending on what ailment is being treated. In that case, the physician could make changes to the settings to enable accessibility for that patient on a scheduled basis for example.

In some implementations, to facilitate a follow-up appointment between a service provider and a person, a code associated with the service provider is provided to the person. Such code can be issued as part of a method of connecting the person to the service provider, especially for follow-up appointments, but could also be for initial appointments as well. The person who has been given a code can enter the code in their client computing device, for example the first client computing device 132, which in turn transmits a message including the code to the server 110. Rather than the server 110 searching for available service providers at steps 203 through 207, the server 110 can attempt to connect the person with the service provider associated with the code. Such method is described in further detail below.

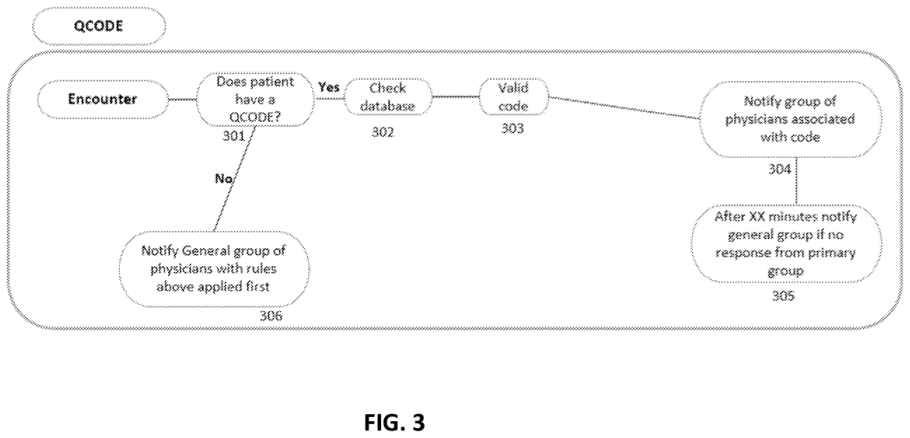

Referring now to FIG. 3, shown is a flowchart of a method of connecting a person to a service provider using a code. In this example, the person is a patient and the service provider is a group of physicians, but the method is similarly applicable to other contexts outside of the medical field. Although the method of FIG. 3 is described below with reference to the server 110 in the communication system 100 shown in FIG. 1, it is to be understood that the method of FIG. 3 is applicable to other systems. In general, the method of FIG. 3 is applicable to the server 110 in any appropriately configured system.

If at step 301 the server 110 receives a code, then at step 302 the server 110 checks the database 120 for that code. If at step 303 the server 110 confirms that the code is valid, then at step 304 the server 110 notifies the service provider associated with the code. In this example, the service provider associated with the code is a primary group of physicians. If the service provider responds within a defined time period, then the person is connected with the service provider. However, if after the defined time period there is no response, then at step 305 the server 110 notifies a general group that the primary group did not respond. The general group can also be notified at step 306 if no code was provided at step 301. A physician from the general group could be found using various rules as similarly described above for steps 203 to 207 of FIG. 2.

There are many possibilities for the code. In specific implementations, the code is a 6-character alphanumeric code. However, more generally, any code that is uniquely linked to a service provider can be used. A code could be generated randomly or assigned. In some implementations, a code would be earmarked to a particular individual or group of individuals depending on parameters set by either an administrator or other (user, Ai?). In some implementations, the code is generated such that it is not easily predictable or hackable for security or privacy reasons. For example, a physician may not want to be contacted by anyone from the general public unless the physician has issued their code. If the code can be easily predicted or hacked, then unauthorised user could use the code to establish appointments with the physician.

In some implementations, the code is generated via an administration panel either with specific access by a group administrator, or by administration. In some implementations, codes can be valid for hours, days, months, specific timeframes, or indefinite periods. In some implementations, codes can be system generated in the case of waitlists for patients to see a doctor. In some implementations, codes are centrally stored and managed, for example in the database 120 of the server 110. In some implementations, upon generation of a code associated with a service provider, the settings for the set of rules are updated to reflect that code, such that the set of rules enable a person who enters that code to be connected to the service provider.

There are many possibilities for the client computing devices 132,134,136,138. The client computing devices 132,134,136,138 can for example include a desktop computer 132, a tablet computer 134, a smartphone 136, a laptop 138, and/or any other appropriate client computing device. Any combination of client computing devices is possible. The client computing devices 132,134,136,138 can communicate with the server 110 using wireless connections as depicted and/or wired connections. Although only four client computing devices 132,134,136,138 are depicted, it is to be understood that there can be more or less than four computing devices.

There are many possibilities for the network 140. The network 140 can include several different networks even though such details are not shown for simplicity. For example, the network 140 can include a RAN (Radio Access Network) for communicating with wireless stations and the Internet for communicating with numerous other computing devices. The network 140 can have other components as well, but these details are not shown for simplicity.

There are many possibilities for the server 110. In some implementations, the server 110 includes a web server and data sent by the server 110 includes web content for a web browser. Additionally, or alternatively, the server 110 can include an application server and data sent by the server 110 includes content for a mobile app. Other implementations are possible.

There are many possibilities for the network adapter 112 of the server 110. In some implementations, the network adapter 112 is a single network adapter 112. In other implementations, the network adapter 112 includes multiple network adapters, for example a first network adapter for communicating with the one or more client computing devices 132,134,136,138, and a second network adapter for communicating with other client computing devices, such as client computing devices utilized by a system administrator. Both wireless and wired network adapters are possible. Any suitable network adapter that can communicate via the network 140 is possible.

There are many possibilities for the rules based engine circuitry 114 of the server 110. In some implementations, the rules based engine circuitry 114 includes a processor 116 that executes software, which can stem from a computer readable medium 118. In some implementations, the computer readable medium 118 also has a database 120 for storing the set of rules and/or other data used by the rules-based engine such as the settings which can be configured. However, other implementations, besides software implementations, are possible and are within the scope of this disclosure. It is noted that other implementations can include additional or alternative hardware components, such as any appropriately configured FPGA (Field-Programmable Gate Array), ASIC (Application-Specific Integrated Circuit), and/or microcontroller, for example. More generally, the rules based engine circuitry 114 of the server 110 can be implemented with any suitable combination of hardware, software and/or firmware.

According to another embodiment of the disclosure, there is provided a non-transitory computer readable medium having recorded thereon statements and instructions that, when executed by the processor 116 of the server 110, implement a method as described herein. The non-transitory computer readable medium can be the computer readable medium 118 of the server 110 shown in FIG. 2, or some other non-transitory computer readable medium. The non-transitory computer readable medium can for example include an SSD (Solid State Drive), a hard disk drive, a CD (Compact Disc), a DVD (Digital Video Disc), a BD (Blu-ray Disc), a memory stick, or any appropriate combination thereof. The non-transitory computer readable medium can include encrypted data at rest.

### Further Details and Case Examples

A first part of a distribution process is when a patient clicks “See Doctor Now”, every suitable physician on the platform that is available gets a text message that there is a patient to be seen and the first physician to answer the text message with an “A” (or other acceptance indication) gets the visit and any accompanying money that goes with it. This create some competition for visits for those that are motivated by money.

Note that it is possible that only a subset of the physician on the platform receive the text message. In some implementations, only suitable physicians on the platform receive the text message. It may not be appropriate to have a doctor connect with a patient that they can't help, so only those physicians that can see the patient receive the text message. For example, for a pediatrician, an age range can be set from 0-20 so as to be notified only for patients between ages of 0-20 that are looking to see a physician. Some GPs may set their age at 5-150, so they will not get a text message if a patient is under the age of 5. Other demographic information such as sex is possible as well. For example, there is little point in sending a male patient to see an Obstetrician/Gynecologist. Thus, sex can be used as a filter. Presenting complaint (i.e. medical issue) can also be used as criteria as well. Another example is by entrance complaint—It would not be appropriate to send anything but eye complaint to an ophthalmologist or anything but a rash to a Dermatologist.

Using a number of key data points such as patient location, reason for visit, age of patient, prior encounter data, time of day, length of time to wait as data input points to the rules based engine. The rules based engine implements an algorithm that will look at factors such as physician location, physician speciality, physician availability (episodic), physician availability (scheduled future clinic), and/or physician previous contact, and select the correct initial contact physician. Once the contact physician has had a predetermined interval to take the call but has not responded, the rules based engine will move into the second physician selection based on appropriate rules. Once this second attempt expires, the call goes out to all notifiable physicians to take the call, in the event that all physicians are currently engaged, the system will automatically offer the patient the option to continue to wait, or opt for a call back by a physician at a pre determined time. The key components encompassing this design are patient, physician input, database, rules based engine, programming of rules based engine, AI (Artificial Intelligence), Rest (Representational state transfer) interfaces.

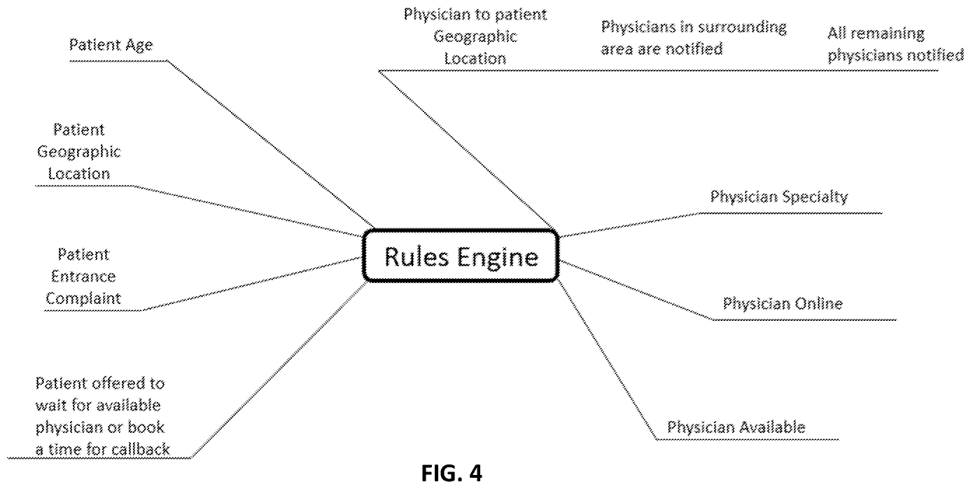

Referring now to FIG. 4, shown is a schematic of a rules based engine for connecting patients to physicians in real-time. The rules based engine addresses assignment and matching of patients and physicians based on a number of key indicators as shown. It is to be understood that the rules engine is very specific and is provided merely as an example. Other rules based engines are possible and are within the scope of the disclosure.

In some implementations, the software is part of a SAAS (Software as a Service) offering. In some implementations, the software runs in AWS (Amazon Web Services) Canada for Canadian operation cloud services. In some implementations, the system operates in the AWS elastic cloud and runs within the AWS Amplify framework. In some implementations, a variety of custom Lambdas are used which are coded in python and JavaScript. External libraries are typically run in an EC2 instance running Ubuntu Linux 16.4. In some implementations, data is encrypted at rest to protect patient level data. In some implementations, the software performs cryptography or otherwise contains components for performing various information security functions (e.g., to protect data during storage or communication, to protect patient data.

One goal is to automatically do what governments have wanted for years: connect a patient to an appropriate health care provider in a timely manner. To do this, a work distribution process is disclosed that takes data from the patient side such as location, age, sex, prior encounter, and/or type of problem they are presenting with (i.e. medical issue), and matches them with a physician that can take care of that type of visit. Not only that, but for a patient not to wait too long, the work distribution process starts with an preferred match and then if the preferred match is not available, it starts looking for other physicians that can provide the care (again using an algorithm). Case examples on how this would work are provided below.

Case #1: A 5 y.o. From Neepawa Presents with a Skin Rash
System looks for a pediatric dermatologist that works in the Neepawa area if none found then goes on,
System looks for a pediatric dermatologist that works in the Prairie Mountain Health region
System looks for a pediatric dermatologist that works in Manitoba
System looks for a dermatologist that works in the Neepawa area
System looks for a dermatologist that works in the Prairie Mountain Health region
System looks for a dermatologist that works in Manitoba
System looks for a pediatrician with an interest in dermatology that works in the Neepawa area
System looks for a pediatrician with an interest in dermatology that works in the Prairie Mountain Health region
System looks for a pediatrician with an interest in dermatology that works in Manitoba
System looks for a pediatrician that works in the Neepawa area
System looks for a pediatrician that works in the Prairie Mountain Health region
System looks for a pediatrician that works in Manitoba
System looks for a GP that sees children that works in the Neepawa area
System looks for a GP that sees children that works in the Prairie Mountain Health region
System looks for a GP that sees children that works in Manitoba

We set time frames between each of the steps to allow for physicians to answer. In most cases we won't need this many steps. The following is the a more likely scenario regarding a visit:

Case #2: An 18 y.o. With a Cough from Neepawa
System looks for a physician that sees 18 y.o. patients that works in the Neepawa area
System looks for a physician that sees 18 y.o. patients that works in the Prairie Mountain Health region
System looks for a physician that sees 18 y.o. patients that works in Manitoba

This could have a time frame of 2 minutes between escalations, but other time frames are possible.

We also see this work distribution process potentially working in other industries (not the matching but the virtually platform and uber distribution process potentially modified so the practitioner sends back availability and the client prepays and accepts the time slots). There is some interest in Family Law, Veterinary medicine etc. An exciting industry would be in education where we could match the appropriate student to the appropriate teacher.

For applications outside medicine, when a person is looking for a service, they could go onto a site, specify what service they want provided, how much they want to spend, how far they want the software to look geographically, and other criteria. This would then put out a notification to service providers that match those criteria in an uber like fashion with those responding first most likely getting the service. Examples are provided below.

Case #3: Student Having Trouble with Chemistry

A grade 10 student is having trouble with chemistry particularly acid base equations—they would be matched in the language of their choice to a teacher that can answer their question and tutor them for as little as 15 minutes on the problem they are having. We would use the same work distribution algorithm trying to match the correct professor or teacher with the student. In this case we may add a monetary component or rating component to the matching.

### Case #4: Person Wants a Men's Haircut

Let's presume a person wants a men's haircut, within 2 hours, at a cost of no more than $30, and with travel no more than 5 km. A request message including personal information is sent, and the system can send out an alert or offer message to those service providers that meet the above criteria and they can respond. In this case, the personal information identifies a desire for haircut as a personal issue, and each service provider is a barber or other hair cut professional.

Case #5: Person Wants an Oil Change for their Car

Let's presume a person wants an oil change for their car. The person specifies the type of car, max price willing to spend, how far they are willing to travel, and when they want the oil change. A request message including personal information is sent, and an offer message goes out to notify service providers. In this case, the personal information identifies a desire for an oil change as a personal issue, and each service provider is an oil changer or other vehicle maintenance provider.

### Examples Scenarios with Codes

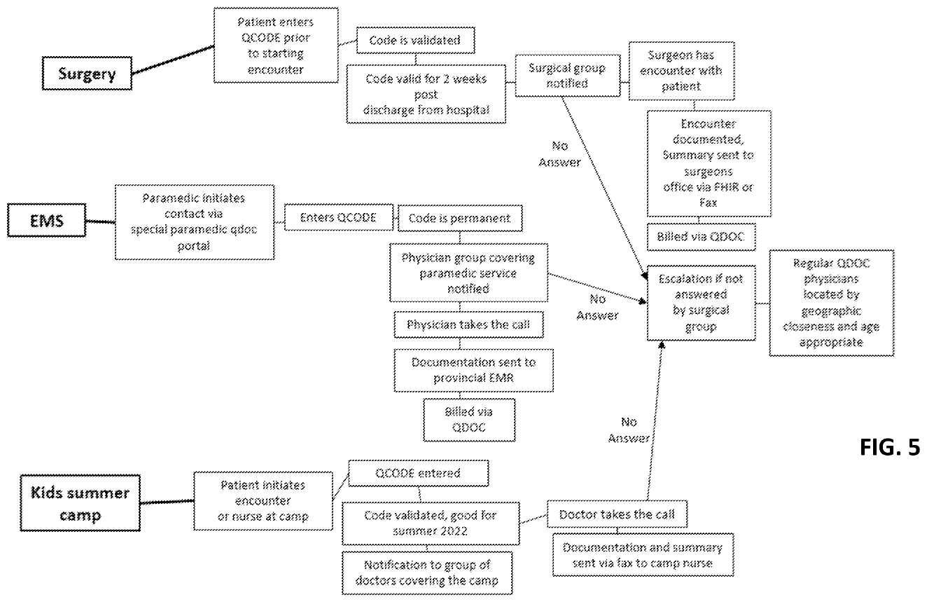

Referring now to FIG. 5, is a schematic of example scenarios of connecting people to service providers using codes. Three example scenarios are shown. It is to be understood that these example scenarios are very specific and are provided merely for exemplary purposes. Other scenarios are possible and are within the scope of the disclosure.

### Case #1: Department of Surgery

A patient is discharged from a hospital, and follow-up care from surgeon may be prudent. The patient logs into the system and initiates an encounter by entering the code provided at time of discharge from the hospital. In this case, the code expires in three weeks. The code is entered and as a result only the group of surgeons responsible for generating the code within the admin console are notified. Surgeon answers call and can now document the encounter and order prescription of follow up as may be appropriate. A summary of the encounter can be sent automatically to the surgeon's office for inclusion in the patients' medical record. If the Surgeon's EMR (Electronic Medical Records) is FHIR (Fast Healthcare Interoperability Resources) compliant, the summary can be sent electronically using the HL7 (Health Level Seven) FHIR standard as opposed to faxing. Billing for the surgeon can be completed via the communication system 100.

### Case #2: EMS Services

A paramedic initiates a patient visit using a paramedic portal. The paramedic begins and encounter with patient using a specific code for their region. This code does not have an expiry date. Call is assigned only to doctors covering this area's paramedic program. The patient is seen. A doctor documents the encounter via the communication system 100 and a copy can be sent automatically to the provincial EMR system via HL7 FHIR or faxing. Billing can be completed via the communication system 100.

### Case #3: Kids Summer Camp

Camps/not for profit/organizations/nursing stations: There are certain groups that may already have physicians covering them, but the coverage is not FT. There are others that may be able to get physicians to cover for then if they didn't have to guarantee FT coverage. The communication system 100 is offering to connect these groups to their physician cohort first and then if their physician group is busy or unable to accommodate the request at a specified time period (requested by the group) then then software would escalate to a different group.

For camps, let's presume a physician has agreed to cover for medical care for two summer camps. Other physicians are joining for one or the other camp. If camp A logs on to the communication system 100, they put it a code which will look for physician group A, if there is no connection made by 10 minutes then the software escalates to any physician. If camp B logs and puts in the appropriate code, then the software looks for physician group B and then may escalate after 15 minutes to all physicians. There may be overlap in the physician groups. For example, a first doctor may be in Camp group A and camp Group B and Downtown community safety partnership group. The software would look for the first doctor (as well as other physicians in a particular group) if any of the corresponding codes for those groups are placed into the software.

For nursing stations, each nursing station may have a code which will tell our software to look for care providers in the following way: a physician covering that nursing station, physicians in that physicians call group, all physicians that can provide care.

Numerous modifications and variations of the present disclosure are possible in light of the above teachings. It is therefore to be understood that within the scope of the appended claims, the disclosure may be practised otherwise than as specifically described herein.

We claim:

1. A method for execution by a server for connecting people with service providers in real-time, comprising:
maintaining a set of rules for connecting people with service providers;
receiving, from a first client computing device over a network, a request message comprising personal information identifying at least a personal issue;
assessing the personal information against a set of rules to identify a first subset of service providers that can address the personal issue;
for each service provider of the first subset, sending an offer message over the network to a client computing device of the service provider; and
if an acceptance message is not received within a first timeout period:
(a) identifying, based on the set of rules, a subsequent subset of service providers that can address the personal issue, such that the subsequent subset includes at least one additional service provider not included in any preceding subset for the request message;
(b) for each additional service provider of the subsequent subset, sending a subsequent offer message over the network to a client computing device of the additional service provider;
(c) if an acceptance message is received within a subsequent timeout period, sending a response message to the first client computing device over the network, such that the response message indicates acceptance.

2. The method of claim 1, further comprising:
if an acceptance message is received over the network within the first timeout period, sending a response message to the first client computing device over the network, such that the response message indicates acceptance.

3. The method of claim 1, further comprising:
if an acceptance message is not received within the subsequent timeout period, repeating steps (a) and (b) until an acceptance message is received within the subsequent timeout period.

4. The method of claim 1, wherein the subsequent subset includes at least one repeat service provider included in any preceding subset, and step (b) comprises sending the subsequent offer message to each service provider of the subsequent subset including each repeat service provider.

5. The method of claim 1, wherein the personal information further identifies a location, and wherein the location is used to identify the first subset of service providers and each subsequent subset of service providers.

6. The method of claim 1, wherein the personal information further identifies timing for service, and wherein the timing for service is used to identify the first subset of service providers and each subsequent subset of service providers.

7. The method of claim 1, wherein each offer message and each acceptance message are text messages.

8. The method of claim 1, wherein if multiple acceptance messages are received in connection with multiple service providers, a first acceptance message of the multiple acceptance messages determines which service providers is selected.

9. The method of claim 1, further comprising:
upon selecting a given service provider, sending, over the network to the client computing device of the given service provider, an acknowledgment message confirming their acceptance of offer.

10. The method of claim 1, wherein the personal information is patient information with the personal issue being a medical issue, and each service provider is a physician or other medical professional.

11. The method of claim 10, wherein the set of rules comprises criteria based on location, age, sex, prior encounter, and medical issue.

12. The method of claim 1, wherein the personal information is student information with the personal issue being an academic issue, and each service provider is a tutor or other teaching professional.

13. The method of claim 12, wherein the set of rules comprises criteria based on location, academic level, and academic issue.

14. The method of claim 1, wherein the set of rules comprise settings, and the method further comprises:
receiving input for modifying the settings; and
updating the settings in accordance with the input.

15. The method of 14, comprising:
updating the settings reflect a code associated with a service provider, such that the set of rules enable a person who enters that code to be connected to that service provider.

16. The method of claim 1, wherein the request message is a first request message for an initial appointment, and the method further comprises:
receiving a second request message comprising a code for a subsequent appointment; and
attempting to connect with a service provider associated with that code.

17. A method for execution by a server for connecting people with service providers in real-time, comprising:
receiving, from a first client computing device over a network, a request message comprising a code;
identifying a service provider associated with the code;
sending an offer message over the network to a client computing device of the service provider; and
if an acceptance message is received over the network within a timeout period, sending a response message to the first client computing device over the network, such that the response message indicates acceptance.

18. The method of claim 17, further comprising:
maintaining a set of rules for connecting people with service providers;
if an acceptance message is not received within the timeout period, attempting to connect with another service provider in accordance with the set of rules.

19. A non-transitory computer readable medium having recorded thereon statements and instructions that, when executed by a processor of a server, configure the server to implement the method of claim 1.

20. A server configured to connect people with service providers in real-time, comprising:
a network adapter; and
rules engine circuitry coupled to the network adapter and configured to implement the method of claim 1.

21. The server of claim 20, wherein:
the rules engine circuitry comprises a processor; and
the server further comprises a non-transitory computer readable medium having recorded thereon statements and instructions that, when executed by the processor, configures the processor as the rules engine circuitry.

22. The server of claim 21, wherein the non-transitory computer readable medium stores the set of rules for connecting people with service providers.
